(19) United States
(12) Patent Application Publication
(10) Pub. No.: US 2017/0326188 A1
(43) Pub. Date: Nov. 16, 2017

(54) USES OF PORIA COCOS EPIDERMIS EXTRACT, PORICOIC ACID A, AND PORICOIC ACID B IN REGULATING BLOOD GLUCOSE LEVEL

(71) Applicant: SINPHAR PHARMACEUTICAL CO., LTD., Yilan County (TW)

(72) Inventors: Hang-Ching LIN, Yilan County (TW); Han-Peng KUO, Yilan County (TW)

(21) Appl. No.: 15/591,261

(22) Filed: May 10, 2017

Related U.S. Application Data
(60) Provisional application No. 62/334,301, filed on May 10, 2016.

(30) Foreign Application Priority Data
May 9, 2017 (TW) 106115288

(51) Int. Cl. A61K 36/076; A61K 31/194; A61K 31/575

(52) U.S. Cl. CPC A61K 36/076; A61K 31/575; A61K 31/194; A61K 2236/39; A61K 2236/333

(57) ABSTRACT

A method for regulating blood glucose level is provided. The method comprises administering to a subject in need an effective amount of at least one of poricoic acid A and poricoic acid B, wherein at least one of poricoic acid A and poricoic acid B can be used in the form of a plant extract, and wherein in the plant extract, poricoic acid A and poricoic acid B are present in a total amount of at least 30 wt %, based on the total weight of the plant extract.

### FIELD OF THE INVENTION

The present invention relates to the uses of Poria cocos epidermis extract, poricoic acid A, and poricoic acid B. The invention especially relates to the uses of Poria cocos epidermis extract, poricoic acid A, and poricoic acid B in regulating blood glucose level.

### BACKGROUND OF THE INVENTION

Diabetes mellitus is a chronic metabolic disorder and is primarily resulted from the malfunction of the mechanism relating to cells' uptake of glucose in an organism, which leads to an overly high level of glucose in the blood. Generally, insulin secreted by pancreatic β cells can stimulate glucose uptake of adipocytes and myocytes, and thus, is effective in regulating blood glucose level. Insufficient insulin secretion in bodies or poor insulin sensitivity due to obesity, aging and other factors in an organism may cause increases in blood glucose level. Hyperglycemia may cause various complications such as hypertension, heart diseases, arteriosclerosis and hyperlipidemia. Severe hyperglycemia may further cause sequelae such as blindness, impotence, amputation, kidney dialysis, etc.

Currently, the method for treating diabetes mellitus in clinic mainly includes exercise, diet control, and drug treatment. Drug treatment includes insulin injection, oral hypoglycemic drugs such as sulfonylureas, biguanides, a-glucosidase inhibitors, insulin sensitizers, etc. However, with the change of human life style, the prevalence of diabetes mellitus has increased yearly. According to the predictions from the World Health Organization in 2008, it is expected that in 2030, there will be over 300 million people with diabetes mellitus globally. Thus, people are still making efforts in developing a drug or method for reducing blood glucose level effectively with few side effects.

Inventors of the present invention found that Poria cocos epidermis extract, as well as poricoic acid A and poricoic acid B contained therein are all effective in promoting the glucose uptake capability of cells, and thus, can be used for regulating blood glucose level, especially for providing an excellent effect on reducing blood glucose level.

### SUMMARY OF THE INVENTION

An objective of the present invention is to provide a use of at least one of poricoic acid A and poricoic acid B in the manufacture of a medicament or a food product, wherein the medicament or food product is for regulating blood glucose level. Based on the total weight of poricoic acid A and poricoic acid B, the medicament or food product is administered at a daily amount ranging from about 0.05 mg/kg-body weight to 1 mg/kg-body weight. The food product could be a health food, a nutritional supplement food, or a special nutritional food.

Preferably, at least one of poricoic acid A and poricoic acid B is used as a plant extract. In the plant extract, poricoic acid A and poricoic acid B are present in a total amount of at least 30 wt %, and preferably at least 40 wt %, based on the total weight of the plant extract. More preferably, at least one of poricoic acid A and poricoic acid B is used as a Poria cocos epidermis extract. In the Poria cocos epidermis extract, each of pachymic acid, dehydropachymic acid, tumulosic acid, and dehydrotumulosic acid is present at an amount of no more than 0.5%, and each of dehydrotrametenolic acid, trametenolic acid, dehydroeburicoic acid, and eburicoic acid is present at an amount of no more than 5%, based on the total weight of the Poria cocos epidermis extract. Preferably, in the Poria cocos epidermis extract, each of dehydrotrametenolic acid, trametenolic acid, dehydroeburicoic acid, and eburicoic acid is present at an amount of no more than 2.5%, based on the total weight of the Poria cocos epidermis extract. More preferably, in the Poria cocos epidermis extract, each of dehydrotrametenolic acid, trametenolic acid, dehydroeburicoic acid, and eburicoic acid is present at an amount of no more than 1%, based on the total weight of the Poria cocos epidermis extract.

Another objective of the present invention is to provide a method for regulating blood glucose level, comprising administering to a subject in need an effective amount of at least one of poricoic acid A and poricoic acid B.

Still another objective of the present invention is to provide a composition for regulating blood glucose level, wherein the composition is a medicament or a food product comprising an effective amount of at least one of poricoic acid A and poricoic acid B.

### BRIEF DESCRIPTION OF THE DRAWINGS

FIG. 1 illustrates the result of the glucose oxidase assay, showing the effect of Poria cocos epidermis extract on the capability of mouse myocytes to uptake glucose in the medium.

FIG. 2 illustrates the result of the glucose oxidase assay, showing the effect of Poria cocos epidermis extract on the capability of mouse adipocytes to uptake glucose in the medium.

FIG. 3A illustrates the result of the glucose oxidase assay, showing the effect of poricoic acid A on the capability of mouse myocytes to uptake glucose in the medium.

FIG. 3B illustrates the result of the glucose oxidase assay, showing the effect of poricoic acid B on the capability of mouse myocytes to uptake glucose in the medium.

FIG. 4A illustrates the result of the glucose oxidase assay, showing the effect of poricoic acid A on the capability of mouse adipocytes to uptake glucose in the medium.

FIG. 4B illustrates the result of the glucose oxidase assay, showing the effect of poricoic acid B on the capability of mouse adipocytes to uptake glucose in the medium.

### DESCRIPTION OF THE PREFERRED EMBODIMENTS

The following will describe some of the embodiments of the present invention in detail. However, without departing from the spirit of the present invention, the present invention may be embodied in various embodiments and should not be limited to the embodiments described in the specification. In addition, unless otherwise indicated herein, the expressions “a,” “an,” “the,” or the like recited in the specification of the present invention (especially in the claims) are intended to include both the singular and plural forms. The term “an effective amount” used in this specification refers to the amount of the compound that can effectively promote the glucose uptake capability of cells in a subject when administered to the subject. The term “subject” used in this specification refers to a mammalian, including human and non-human animals. The phrase “regulating blood glucose level” used in this specification refers to changing the concentration of blood glucose towards a normal value. The unit “mg/kg-body weight” used in this specification refers to the dosage required per kg of body weight.

The numerical ranges (e.g., 5 to 100) used in this specification should be construed as including all of the rational numbers in the ranges and ranges consisting of any rational numbers in the ranges. Therefore, the numerical ranges used in this specification should include all the possible combinations of numerical values between the lowest value and the highest value listed therein. In addition, the word “about” as used herein substantially represents values within ±20% of the stated value, preferably within ±10% and more preferably within ±5%.

As described above, diabetes mellitus is primarily resulted from the malfunction of the mechanism relating to cells' uptake of glucose in an organism, which leads to an overly high level of glucose in the blood. Inventors of the present invention found that poricoic acid A and poricoic acid B both are effective in promoting the glucose uptake capability of cells, and thus, can be used for regulating blood glucose level, especially for reducing the overly high blood glucose level. Therefore, the present invention provides the uses of at least one of poricoic acid A and poricoic acid B in regulating blood glucose level, comprising the use of at least one of poricoic acid A and poricoic acid B in the manufacture of a medicament or a food product for regulating blood glucose level, a method for regulating blood glucose level comprising administering at least one of poricoic acid A and poricoic acid B to a subject in need, and a food product or a pharmaceutical composition comprising at least one of poricoic acid A and poricoic acid B.

Herbal FU-LING refers to the dried sclerotium of Poria cocos (Schw.) Wolf, a fungus in the family Fomitopsidaceae. Poria cocos (FU-LING) fungus often parasitizes the roots of pine trees. The external layer of Poria cocos (FU-LING) is of light brown or dark brown color (called as Poria cocos epidermis) and the inside of Poria cocos (FU-LING) is of pink or white color (called as Poria cocos meat). According to traditional Chinese medicine classics, Poria cocos meat is used in sedation, diuresis, nutrient supplementation, immunity enhancement and aging delay, while Poria cocos epidermis is only used in the treatment of skin edema.

As shown in the examples provided hereinafter, according to the present invention, a Poria cocos epidermis extract comprising poricoic acid A and poricoic acid B in a total amount of at least 30 wt % (based on the total weight of the Poria cocos epidermis extract) can be prepared from Poria cocos epidermis. Therefore, at least one of poricoic acid A and poricoic acid B adopted by the present invention could be used as a plant extract such as Poria cocos epidermis extract. In the plant extract adopted by the present invention, poricoic acid A and poricoic acid B are present in a total amount of at least 30 wt %, and preferably at least 40 wt %, based on the total weight of the plant extract.

The Poria cocos epidermis extract adopted by the present invention can be provided by an operation comprising the following steps: (a) extracting a Poria cocos epidermis with a first polar solvent to provide a crude extract; (b) drying the crude extract to provide a crude extract powder; and (c) extracting the crude extract powder with a second polar solvent to provide a Poria cocos epidermis extract. The first polar solvent and the second polar solvent are the same or different and are respectively selected from the group consisting of water, ethanol, basic solution, acidic solution, and combinations thereof. The basic solution refers to any suitable basic solution that has a pH value of more than 7 (e.g., sodium hydroxide solution), and the acidic solution refers to any suitable acidic solution that has a pH value of less than 7 (e.g., hydrochloric acid solution). In some embodiments of the present invention, aqueous ethanol solutions having the same or different ethanol concentrations were used as the first polar solvent and the second polar solvent.

In step (a), the ratio of the amounts of first polar solvent and Poria cocos epidermis could be optionally adjusted. In general, there is no particular limitation to the amount of first polar solvent being used, as long as the materials can be dispersed in the first polar solvent evenly. For example, in step (a), the first polar solvent and Poria cocos epidermis could be used at a volume ratio ranging from about 8:1 to about 16:1 (first polar solvent: Poria cocos epidermis). In one embodiment of the present invention, the extraction of step (a) was carried out with the use of an aqueous ethanol solution as the first polar solvent and at a volume ratio of Poria cocos epidermis:aqueous ethanol solution=1:8.

In step (a), the extraction could be conducted for a suitable period of time depending on the first polar solvent being adopted. For example, when an aqueous ethanol solution is used as the first polar solvent and the volume ratio of Poria cocos epidermis:aqueous ethanol solution is 1:8, the extraction is usually conducted for at least 1 hour, preferably at least 2 hours, and more preferably at least 3 hours. Step (a) could be optionally accompanied with other operations such as decoction, cooling, filtration, vacuum concentration, and resin column chromatography. Optionally, the Poria cocos epidermis could be pre-soaked in the first polar solvent for a period of time prior to conducting step (a). For example, the Poria cocos epidermis could be pre-soaked in the first polar solvent for about 12 hours when an aqueous ethanol solution is served as the first polar solvent.

In step (c), the ratio of the amounts of second polar solvent and the crude extract powder obtained from step (b) could be optionally adjusted. In general, there is no particular limitation to the amount of second polar solvent being used, as long as the crude extract powder can be dispersed in the second polar solvent evenly. For example, in step (c), the second polar solvent and the Poria cocos epidermis crude extract powder could be used at a volume ratio ranging from about 8:1 to about 16:1 (second polar solvent: Poria cocos epidermis crude extract powder). In one embodiment of the present invention, the extraction of step (c) was carried out with the use of an aqueous ethanol solution as the second polar solvent and at a volume of Poria cocos epidermis crude extract powder:aqueous ethanol solution=1:8.

The Poria cocos epidermis extract adopted in accordance with the present invention could be a dry matter, which can be provided by drying the liquid extract obtained from step (c). To achieve an extraction efficiency as high as possible, the extraction of Poria cocos epidermis could be optionally repeated with the same or different first polar solvents prior to step (b), and the liquid extracts thus obtained could be combined to provide the crude extract for use in step (b). Also, the step (b), step (c), and the cycle of other optional operations described above could be repeated.

In the Poria cocos epidermis extract adopted by the present invention, poricoic acid A and poricoic acid B are present in a total amount of at least 30 wt %, and preferably at least 40 wt %, based on the total weight of the Poria cocos epidermis extract: each of pachymic acid, dehydropachymic acid, tumulosic acid, and dehydrotumulosic acid is present at an amount of no more than 0.5%, and each of dehydrotrametenolic acid, trametenolic acid, dehydroeburicoic acid, and eburicoic acid is present at an amount of no more than 5%, based on the total weight of the Poria cocos epidermis extract. Preferably, in the Poria cocos epidermis extract, each of dehydrotrametenolic acid, trametenolic acid, dehydroeburicoic acid, and eburicoic acid is present at an amount of no more than 2.5%, based on the total weight of the Poria cocos epidermis extract. More preferably, in the Poria cocos epidermis extract, each of dehydrotrametenolic acid, trametenolic acid, dehydroeburicoic acid, and eburicoic acid is present at an amount of no more than 1%, based on the total weight of the Poria cocos epidermis extract.

Depending on the desired purpose, the pharmaceutical composition or medicament according to the present invention could be provided in any suitable form without specific limitations. For example, the pharmaceutical composition or medicament could be administered to a subject in need by an oral or parenteral (such as subcutaneous, intravenous, muscular, or peritoneal) route, but the administration is not limited thereby. Depending on the form and purpose, suitable carriers could be chosen and used to provide the pharmaceutical composition or medicament, wherein the carriers include excipients, diluents, auxiliaries, stabilizers, absorbent retarders, disintegrating agent, hydrotropic agents, emulsifiers, antioxidants, adhesives, binders, tackifiers, dispersants, suspending agents, lubricants, hygroscopic agents, etc.

As a dosage form for oral administration, the pharmaceutical composition or medicament provided according to the present invention could comprise any pharmaceutically acceptable carrier that will not adversely affect the desired effects of the active ingredients (i.e., poricoic acid A, poricoic acid B, or Poria cocos epidermis extract). For example, the pharmaceutically acceptable carrier could be water, saline, dextrose, glycerol, ethanol or its analogs, cellulose, starch, sugar bentonite, and combinations thereof. The pharmaceutical composition or medicament could be provided in any suitable form for oral administration, such as in the form of a tablet (e.g., dragee), a pill, a capsule, granules, a pulvis, a fluidextract, a solution, a syrup, a suspension, a tincture, etc.

As for the form of injections or drips suitable for subcutaneous, intravenous, intramuscular, or peritoneal administration, the pharmaceutical composition or medicament provided according to the present invention could comprise one or more ingredient(s), such as an isotonic solution, a salt-buffered saline (e.g., phosphate-buffered saline or citrate-buffered saline), a hydrotropic agent, an emulsifier, 5% sugar solution, and other carriers to provide the pharmaceutical composition or medicament as an intravenous infusion, an emulsified intravenous infusion, a powder for injection, a suspension for injection, or a powder suspension for injection, etc. Alternatively, the pharmaceutical composition or medicament could be prepared as a pre-injection solid. The pre-injection solid could be provided in a form which is soluble in other solutions or suspensions, or in an emulsifiable form. A desired injection is provided by dissolving the pre-injection solid in other solutions or suspensions or emulsifying it prior to being administered to a subject in need.

Optionally, the pharmaceutical composition or medicament provided according to the present invention could further comprise a suitable amount of additives, such as a flavoring agent, a toner, or a coloring agent for enhancing the palatability and the visual perception of the pharmaceutical composition or medicament, and/or a buffer, a conservative, a preservative, an antibacterial agent, or an antifungal agent for improving the stability and storability of the pharmaceutical composition or medicament. In addition, the pharmaceutical composition or medicament could optionally further comprise one or more other active ingredient(s) (such as antioxidants, insulin sensitizers, etc.), or be used in combination with a medicament comprising one or more other active ingredient(s), to further enhance the effects of the pharmaceutical composition or medicament, or to increase the application flexibility and adaptability of the preparation thus provided, as long as the other active ingredients do not adversely affect the desired effects of the active ingredients of the present invention (i.e., poricoic acid A, poricoic acid B, or Poria cocos epidermis extract).

Depending on the need, age, body weight, and health conditions of the subject, the pharmaceutical composition or medicament provided according to the present invention could be dosed with various administration frequencies, such as once a day, multiple times a day, or once every few days, etc. For example, when the pharmaceutical composition or medicament is applied orally to a subject for regulating blood glucose level, the dosage of the pharmaceutical composition or medicament is about 0.01 mg/kg-body weight to about 5 mg/kg-body weight per day, preferably about 0.03 mg/kg-body weight to about 2 mg/kg-body weight per day, and more preferably about 0.05 mg/kg-body weight to about 1 mg/kg-body weight per day, based on the total weight of poricoic acid A and poricoic acid B. Alternatively, the dosage of the pharmaceutical composition or medicament is about 0.025 mg/kg-body weight to about 25 mg/kg-body weight per day, preferably about 0.075 mg/kg-body weight to about 10 mg/kg-body weight per day, and more preferably about 0.125 mg/kg-body weight to about 5 mg/kg-body weight per day, based on the total weight of Poria cocos epidermis extract.

The food product according to the present invention could be a health food, a nutritional supplement food or a special nutritional food. The food product may be provided as dairy products, meat products, breadstuff, pasta, cookies, troche, capsule, fruit juices, teas, sport beverages, nutritional beverages, etc., but is not limited thereby. Preferably, the food product according to the present invention is a health food.

Depending on the recommended daily dosage for the age, body weight and health conditions of the subject, the health food, nutritional supplement food and special nutritional food provided according to the present invention could be taken in various frequencies, such as once a day, multiple times a day or once every few days, etc. The amount of poricoic acid A, poricoic acid B, or Poria cocos epidermis extract in the health food, nutritional supplement food and special nutritional food provided by the present invention could be adjusted, preferably to the amount that should be taken daily, depending on the specific population. For example, if the recommended daily dosage for a subject is about 70 mg of the total weight of poricoic acid A and poricoic acid B per day and each serving of the health food contains poricoic acid A and poricoic acid B at a total amount of 35 mg, the subject can take about two servings of the health food per day.

The recommended daily dosage, use standards and use conditions for a specific population (e.g., pregnant women, diabetes mellitus patients, and kidney disease patients), or the recommendations for a use in combination with another food product or medicament could be indicated on the exterior package of the health food, nutritional supplement food and/or special nutritional food provided by the present invention. Thus, it is suitable for the user to take the health food, nutritional supplement food and/or special nutritional food by him- or herself safely and securely without the instructions of a doctor, pharmacist, or related executive.

The present invention further provides a method for regulating blood glucose level, comprising administering to a subject in need an effective amount of an active ingredient, wherein the active ingredient is at least one of poricoic acid A and poricoic acid B. In the method for regulating blood glucose level according to the present invention, the type, applied route, applied form, suitable dosage and use of the active ingredients in related treatment are all in line with the above description.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

### Preparation Examples

A. Preparation of Poria cocos Epidermis Extract

A-1.

Poria cocos (also called as herbal FU-LING; habitat: Yunnan, China) was washed and its skin was peeled (hereinafter referred to as “Poria cocos epidermis”), and the rest was meat (hereinafter referred to as “Poria cocos meat”). The Poria cocos epidermis was soaked in 75% aqueous ethanol solution (Poria cocos epidermis: 75% aqueous ethanol solution=1:8 in volume) at room temperature for 12 hours, and then decocted for 3 hours to provide a liquid extract. The foregoing extraction procedures were repeated three times. The liquid extracts obtained from the three extractions were combined and filtered to remove the insoluble and provide a crude extract. The solvent contained in the crude extract was removed by vacuum concentration to provide a concentrated solution. The concentrated solution was dried by using a spray dryer to provide a crude extract powder.

A-2.

The crude extract powder obtained from A-1 was extracted with 95% aqueous ethanol solution (crude extract powder: 95% aqueous ethanol solution=1:8 in volume) for 3 hours, and then the liquid extract obtained therefrom was eluted by a column with a stationary phase of silica gel to provide a Poria cocos epidermis extract.

A-3.

The crude extract powder obtained from A-1 was dispersed evenly in pure water (crude extract powder:water=1:10 in volume) to provide a mixture. Then, sodium hydroxide was added into the mixture to increase the pH value of the mixture to about 12. The mixture was poured into a mixing barrel which maintained at 65° C. and was stirred evenly until the reaction was completed. Thereafter, the mixture was neutralized with 12N concentrated hydrochloric acid and then centrifuged to remove the filtrate. The remaining insoluble was washed with pure water, and then dried by using a spray dryer to provide an extract powder. The extract powder was extracted three times with 1N sodium bicarbonate solution (extract powder: 1N sodium bicarbonate solution=1:20 in volume), and the liquid extracts obtained from the three extractions were combined and neutralized with 12N concentrated hydrochloric acid, and then centrifuged to remove the filtrate. The remaining insoluble was washed with pure water, and then dried by using a spray dryer to provide a Poria cocos epidermis extract.

A-4.

The components of extracts obtained from A-2 and A-3 were determined by liquid chromatography coupled to diode array UV detection and mass spectrometer (LC/UV/MS) at 243 nm and 210 nm wavelength respectively, and the content of each component contained in the extracts were quantified by high performance liquid chromatography (HPLC). The results of the extract obtained from A-2 are shown in Table 1, and the results of the extract obtained from A-3 are shown in Table 2.

TABLE 1

| component | wt % |
|---|---|
| Pachymic acid (PA) | — |
| Dehydropachymic acid (DPA) | — |
| Tumulosic acid (TA) | — |
| Dehydrotumulosic acid (DTA) | 0.46 |
| Polyporenic acid C (PAC) | 2.01 |
| 3-epi-dehydrotumulosic acid (EDTA) | 0.77 |
| Dehydrotrametenolic acid (DTTA) | 2.01 |
| Trametenolic acid (TTA) | 0.85 |
| Poricoic acid A (PAA) | 32.72 |
| Dehydroeburicoic acid (DEA) | 1.20 |
| Poricoic acid B (PAB) | 10.44 |
| Eburicoic acid (EA) | 0.83 |

TABLE 2

| component | wt % |
|---|---|
| Pachymic acid (PA) | — |
| Dehydropachymic acid (DPA) | — |
| Tumulosic acid (TA) | — |
| Dehydrotumulosic acid (DTA) | 0.31 |
| Polyporenic acid C (PAC) | 1.15 |
| 3-epi-dehydrotumulosic acid (EDTA) | 0.40 |
| Dehydrotrametenolic acid (DTTA) | 0.58 |
| Trametenolic acid (TTA) | 0.35 |
| Poricoic acid A (PAA) | 41.06 |
| Dehydroeburicoic acid (DEA) | 0.19 |
| Poricoic acid B (PAB) | 16.50 |
| Eburicoic acid (EA) | 0.20 |

As shown in Table 1, the Poria cocos epidermis extract obtained from A-2 has a high content of poricoic acid A (at a weight percentage of 32.72% in the extract) and poricoic acid B (at a weight percentage of 10.44% in the extract), a low content of dehydrotrametenolic acid, trametenolic acid, dehydroeburicoic acid and eburicoic acid (at weight percentages of 2.01%, 0.85%, 1.2% and 0.83% in the extract, respectively), and a very low content of dehydrotumulosic acid (at a weight percentage of 0.46% in the extract), but does not contain pachymic acid, dehydropachymic acid, and tumulosic acid.

As shown in Table 2, the Poria cocos epidermis extract obtained from A-3 has a high content of poricoic acid A (at a weight percentage of 41.06% in the extract) and poricoic acid B (at a weight percentage of 16.50% in the extract), a low content of polyporenic acid C and dehydrotrametenolic acid (at weight percentages of 1.15% and 0.58% in the extract, respectively), and a very low content of dehydrotumulosic acid, 3-epi-dehydrotumulosic acid, trametenolic acid, dehydroeburicoic acid, and eburicoic acid (at weight percentages of 0.31%, 0.40%, 0.35%, 0.19% and 0.20% in the extract, respectively), but does not contain pachymic acid, dehydropachymic acid, and tumulosic acid.

B. Preparations of Poricoic Acid a and Poricoic Acid B

B-1.

The Poria cocos epidermis extract obtained from A-2 or A-3 was dispersed evenly in methanol (Poria cocos epidermis extract:methanol=1:500 in volume) to provide a mixture. Then, the mixture was filtrated to remove the insoluble. The remaining filtrate was purified by preparative high performance liquid chromatography (with a mobile phase of a mixture of methanol and water) at 243 nm wavelength, and the portion of poricoic acid A and poricoic acid B containing was collected. The collected portion was vacuum-concentrated to remove methanol and obtain poricoic acid A and poricoic acid B respectively.

B-2.

The poricoic acid A and poricoic acid B obtained from B-1 were detected by liquid chromatography coupled to diode array UV detection and mass spectrometer (LC/UV/MS) at 243 nm wavelength, and the results show that the purities of poricoic acid A and poricoic acid B were all higher than 98%.

C. Cell Cultivation

The differentiated mouse myocytes (C2C12, purchased from ATCC) and adipocytes (3T3-L1, purchased from ATCC) were cultivated in a Dulbecco's modified Eagle's medium (DMEM) containing 2% bovine serum albumin (BSA) but serum free for 16 hours respectively. Then, the cells were washed with Dulbecco's phosphate-buffered saline (D-PBS), and medium was replaced with a 0.2% BSA-DMEM medium containing 500 μg/ml glucose. The cells thus provided were used in the following experiments.

### Example 1: Effects of Poria cocos Epidermis Extract on the Glucose Uptake Capability of Cells

(1-1) Myocytes

C2C12 cells provided by [Preparation Examples] were divided into 5 groups and were respectively cultivated with the following mediums for 2 hours:
1. Group I: a BSA-DMEM medium containing 500 μg/ml glucose.
2. Group II: a BSA-DMEM medium containing 500 μg/ml glucose and 100 nM insulin.
3. Group III: a BSA-DMEM medium containing 500 μg/ml glucose and 0.1 μg/ml Poria cocos epidermis extract provided by [Preparation Example A-2].
4. Group IV: a BSA-DMEM medium containing 500 μg/ml glucose and 1 μg/ml Poria cocos epidermis extract provided by [Preparation Example A-2].
5. Group V: a BSA-DMEM medium containing 500 μg/ml glucose and 10 μg/ml Poria cocos epidermis extract provided by [Preparation Example A-2].

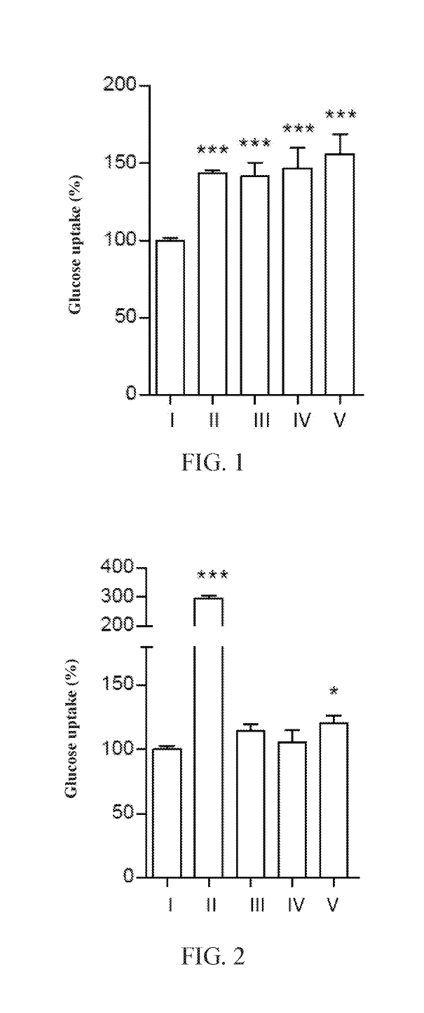

Thereafter, the content of glucose in the medium of each group was determined by a glucose oxidase assay to measure the level of glucose consumption (representing the glucose uptake capability of cells) of each group. Finally, the results of the control group (i.e., cells being cultivated with the medium of Group I) was served as a basis to calculate the relative glucose uptake capabilities of other groups. The results are shown in FIG. 1.

As shown in FIG. 1, as compared to the control group, the glucose uptake capability of cells being treated with the Poria cocos epidermis extract of the present invention (i.e., cells being cultivated with the medium of Group III, IV or V) significantly increased, and even exceeded that of the positive control group (i.e., cells being cultivated with the medium of Group II). These results indicate that Poria cocos epidermis extract of the present invention is effective in promoting the glucose uptake capability of myocytes, and thus, can be used for regulating blood glucose level.

(1-2) Adipocytes

3T3-L1 cells provided by [Preparation Examples] were divided into 5 groups and were respectively cultivated with the mediums of Groups I to V described in Example 1 (1-1) for 2 hours. Then, the content of glucose in the medium of each group were determined by a glucose oxidase assay to measure the level of glucose consumption of each group (representing the glucose uptake capability of cells). Finally, the results of the control group (i.e., cells being cultivated with the medium of Group I) was served as a basis to calculate the relative glucose uptake capabilities of other groups. The results are shown in FIG. 2.

As shown in FIG. 2, as compared to the control group, the cells being treated with the Poria cocos epidermis extract of the present invention (i.e., cells being cultivated with the medium of Group III, IV or V) exhibited an increment in the glucose uptake capability, and the increment was especially significant for the high dosage group (i.e., cells being cultivated with the medium of Group V). These results indicate that Poria cocos epidermis extract of the present invention is effective in promoting the glucose uptake capability of adipocytes, and thus, can be used for regulating blood glucose level.

### Example 2: Effects of Poricoic Acid A and Poricoic Acid B on the Glucose Uptake Capability of Cells

(2-1) Myocytes

C2C12 cells provided by [Preparation Examples] were divided into 8 groups and were respectively cultivated with the following mediums for 2 hours:
1. Group i: a BSA-DMEM medium containing 500 μg/ml glucose.
2. Group ii: a BSA-DMEM medium containing 500 μg/ml glucose and 100 nM insulin.
3. Group iii: a BSA-DMEM medium containing 500 μg/ml glucose and 0.1 μg/ml poricoic acid A or poricoic acid B provided by [Preparation Example B-1].
4. Group iv: a BSA-DMEM medium containing 500 μg/ml glucose and 1 μg/ml poricoic acid A (or poricoic acid B) provided by [Preparation Example B-1].
5. Group v: a BSA-DMEM medium containing 500 μg/ml glucose and 10 μg/ml poricoic acid A (or poricoic acid B) provided by [Preparation Example B-1].

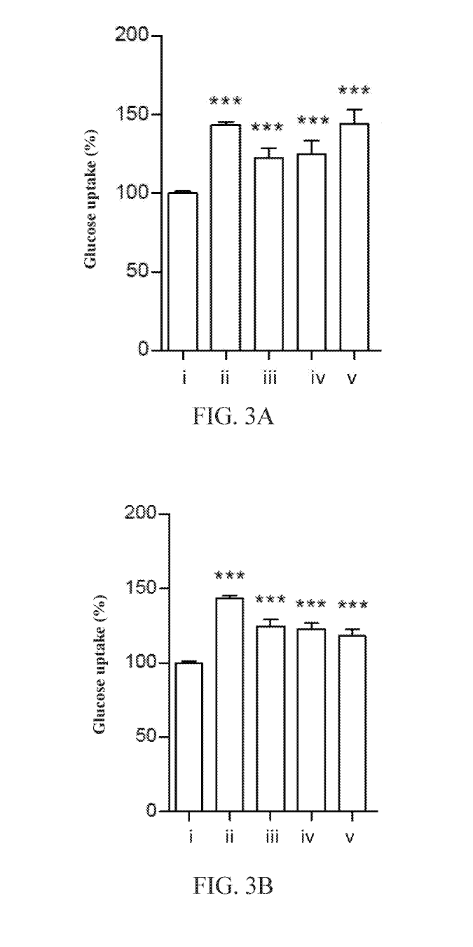

Then, the content of glucose in the medium of each group was determined by a glucose oxidase assay to measure the level of glucose consumption of each group (representing the glucose uptake capability of cells). Finally, the results of the control group (i.e., cells being cultivated with the medium of Group i) was served as a basis to calculate the relative glucose uptake capabilities of other groups. The results are shown in FIGS. 3A and 3B. FIG. 3A comprises the results of the control group, the positive control group (i.e., cells being cultivated with the medium of Group ii), and the groups being treated with poricoic acid A (i.e., cells being cultivated with the medium of Group iii, iv or v which contains poricoic acid A). FIG. 3B comprises the results of the control group, the positive control group, and the groups being treated with poricoic acid B (i.e., the cells being cultivated with the medium Group iii, iv or v which contains poricoic acid B).

As shown in FIG. 3A, as compared to the control group, the glucose uptake capabilities of the groups being treated with poricoic acid A significantly increased to be equivalent to that of the positive control group. These results indicate that poricoic acid A is effective in promoting the glucose uptake capability of myocytes, and thus, can be used for regulating blood glucose level.

As shown in FIG. 3B, as compared to the control group, the glucose uptake capabilities of the groups being treated with poricoic acid B also significantly increased. These results indicate that poricoic acid B can also regulate blood glucose level by promoting the glucose uptake capability of myocytes.

(2-2) Adipocytes

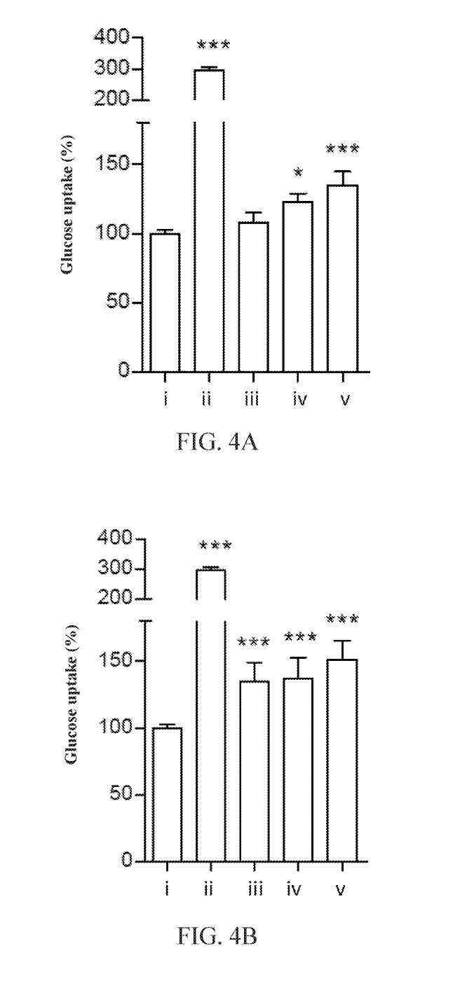

3T3-L1 cells provided by [Preparation Examples] were divided into 8 groups and were respectively cultivated with the mediums of Groups i to v as described in Example 2 (2-1) for 2 hours. Then, the content of glucose in the medium of each group was determined by a glucose oxidase assay to measure the level of glucose consumption of each group (representing the glucose uptake capability of cells). Finally, the results of the control group (i.e., cells being cultivated with the medium of Group i) was served as a basis to calculate the relative glucose uptake capabilities of other groups. The results are shown in FIGS. 4A and 4B. FIG. 4A comprises the results of the control group, the positive control group (i.e., cells being cultivated with the medium of Group ii), and the groups being treated with poricoic acid A (i.e., cells being cultivated with the medium of Group iii, iv or v which contains poricoic acid A). FIG. 4B comprises the results of the control group, the positive control group, and the group being treated with poricoic acid B (i.e., cells being cultivated with the medium of Group iii, iv or v which contains poricoic acid B).

As shown in FIG. 4A, as compared to the control group, the groups being treated with poricoic acid A exhibited an increment in the glucose uptake capabilities and the increment was significant for the medium dosage group (i.e., cells being cultivated with the medium of Group iv) and high dosage group (i.e., cells being cultivated with the medium of Group v). These results indicate that poricoic acid A is effective in promoting the glucose uptake capability of adipocytes, and thus, can be used for regulating blood glucose level.

As shown in FIG. 4B, as compared to the control group, the glucose uptake capabilities of the groups being treated with poricoic acid B significantly increased. These results indicate that poricoic acid B can also regulate blood glucose level by promoting the glucose uptake capability of adipocytes.

As the aforementioned examples, Poria cocos epidermis extract, poricoic acid A, and poricoic acid B of the present invention indeed can promote the glucose uptake capability in an organism, and thus, can be used for regulating blood glucose level, especially for reducing the overly high blood glucose level.

### BRIEF DESCRIPTION OF REFERENCE NUMERALS

Not applicable.

### DEPOSIT OF BIOLOGICAL MATERIAL

Not applicable.

### SEQUENCE LISTING

Not applicable.

What is claimed is:

1. A method for regulating blood glucose level, comprising administering to a subject in need an effective amount of at least one of poricoic acid A and poricoic acid B.

2. The method as claimed in claim 1, wherein at least one of poricoic acid A and poricoic acid B is used as a plant extract.

3. The method as claimed in claim 2, wherein in the plant extract, poricoic acid A and poricoic acid B are present in a total amount of at least 30 wt %, based on the total weight of the plant extract.

4. The method as claimed in claim 3, wherein in the plant extract, poricoic acid A and poricoic acid B are present in a total amount of at least 40 wt %, based on the total weight of the plant extract.

5. The method as claimed in claim 2, wherein the plant extract is a Poria cocos epidermis extract.

6. The method as claimed in claim 5, wherein in the Poria cocos epidermis extract, each of pachymic acid, dehydropachymic acid, tumulosic acid, and dehydrotumulosic acid is present at an amount of no more than 0.5%, based on the total weight of the Poria cocos epidermis extract.

7. The method as claimed in claim 5, wherein in the Poria cocos epidermis extract, each of dehydrotrametenolic acid, trametenolic acid, dehydroeburicoic acid, and eburicoic acid is present at an amount of no more than 5%, based on the total weight of the Poria cocos epidermis extract.

8. The method as claimed in claim 7, wherein in the Poria cocos epidermis extract, each of dehydrotrametenolic acid, trametenolic acid, dehydroeburicoic acid, and eburicoic acid is present at an amount of no more than 2.5%, based on the total weight of the Poria cocos epidermis extract.

9. The method as claimed in claim 8, wherein in the Poria cocos epidermis extract, each of dehydrotrametenolic acid, trametenolic acid, dehydroeburicoic acid, and eburicoic acid is present at an amount of no more than 1%, based on the total weight of the Poria cocos epidermis extract.

10. The method as claimed in claim 5, wherein the Poria cocos epidermis extract is provided by an operation comprising the following steps:
(a) extracting a Poria cocos epidermis with a first solvent to provide a crude extract;
(b) drying the crude extract to provide a crude extract powder; and
(c) extracting the crude extract powder with a second solvent to provide a Poria cocos epidermis extract,
wherein the first solvent and the second solvent are the same or different and are respectively selected from the group consisting of water, ethanol, basic solution, acidic solution, and combinations thereof.

11. The method as claimed in claim 10, wherein each of the first solvent and the second solvent is an aqueous ethanol solution having the same or different ethanol concentration.

12. The method as claimed in claim 1, wherein based on the total weight of poricoic acid A and poricoic acid B, the poricoic acid A and poricoic acid B are administered to the subject at a daily amount ranging from about 0.05 mg/kg-body weight to 1 mg/kg-body weight.
